(19) United States
(12) Patent Application Publication
(10) Pub. No.: US 2010/0009906 A1
(43) Pub. Date: Jan. 14, 2010

(54) Anticancer Treatments

(75) Inventor: Yusri Ali Elsayed, Hillsborough, NJ (US)

Correspondence Address: KING & SPALDING, 1185 AVENUE OF THE AMERICAS, NEW YORK, NY, 10036-4003, US

(73) Assignee: PHARMA MAR, S.A., Madrid (ES)

(21) Appl. No.: 12/299,960

(22) PCT Filed: May 11, 2007
(86) PCT No.: PCT/US07/68727

§ 371 Date: Mar. 23, 2009

Related U.S. Application Data
(60) Provisional application No. 60/800,510, filed on May 12, 2006.

(51) Int. Cl. A61K 38/19; A61K 31/4995; A61P 35/00

(52) U.S. Cl. 514/12; 514/250

(57) ABSTRACT

A method of treating the human body for cancer comprises administering an effective therapeutic amount of docetaxel in combination with an effective therapeutic amount of ET-743.

This application claims benefit of priority from U.S. 60/800,510, filed May 12, 2006, the contents of which are hereby incorporated by reference.

The present invention relates to the treatment of cancers and, in particular, an effective treatment of human cancers using Ecteinascidin 743 (ET-743) in combination with another drug.

### BACKGROUND OF THE INVENTION

Cancer develops when cells in a part of the body begin to grow out of control. Although there are many kinds of cancer, they all start because of out-of-control growth of abnormal cells. Cancer cells can invade nearby tissues and can spread through the bloodstream and lymphatic system to other parts of the body. There are several main types of cancer. Carcinoma is cancer that begins in the skin or in tissues that line or cover internal organs. Epithelial cells, which cover internal and external surfaces of the body, including organs and lining of vessels, may give rise to a carcinoma. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system.

In addition, cancer is invasive and tends to metastasise to new sites. It spreads directly into surrounding tissues and also may be disseminated through the lymphatic and circulatory systems.

Many treatments are available for cancer, including surgery and radiation for localised disease, and chemotherapy. However, the efficacy of available treatments for many cancer types is limited, and new, improved forms of treatment showing clinical benefit are needed. This is especially true for those patients presenting with advanced and/or metastatic disease and for patients relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment and ultimately die from progressive metastatic disease. Thus, the ongoing commitment to the design and discovery of new anticancer agents is critically important. Chemotherapy, in its classic form, has been focused primarily on killing rapidly proliferating cancer cells by targeting general cellular metabolic processes, including DNA, RNA, and protein biosynthesis.

Chemotherapy drugs are divided into several groups based on how they affect specific chemical substances within cancer cells, which cellular activities or processes the drug interferes with, and which specific phases of the cell cycle the drug affects. The most commonly used types of chemotherapy drugs include: DNA-alkylating drugs (such as cyclophosphamide, ifosfamide, cisplatin, carboplatin, dacarbazine), antimetabolites (5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase II inhibitors (such as topotecan, irinotecan, etoposide, teniposide), and hormone therapy (such as tamoxifen, flutamide). The ideal antitumor drug would kill cancer cells selectively, with a wide index relative to its toxicity towards non-cancer cells and it would also retain its efficacy against cancer cells, even after prolonged exposure to the drug. Unfortunately, none of the current chemotherapies with these agents posses an ideal profile. Most posses very narrow therapeutic indexes and, in addition, cancerous cells exposed to slightly sublethal concentrations of a chemotherapeutic agent may develop resistance to such an agent, and quite often cross-resistance to several other antitumor agents.

Ecteinascidin 743 (ET-743) is a tetrahydroisoquinoline alkaloid originally isolated from the marine tunicate Ecteinascidia turbinate with the following structure:

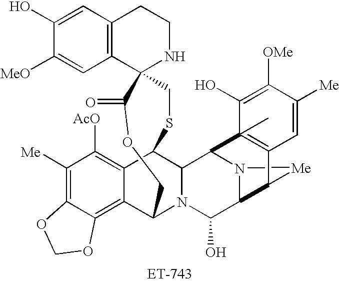

A review of ET-743, its chemistry, mechanism of action and preclinical and clinical development can be found in Kesteren, Ch. Van et al., 2003, Anti-Cancer Drugs, 14 (7), pages 487-502: “Yondelis (trabectedin, ET-743): the development of an anticancer agent of marine origin”, and references therein.

ET-743 possesses potent anticancer activity against a variety of human tumour xenografts grown in athymic mice, including, for example, melanoma, ovarian and breast carcinoma.

Promising ET-743 responses have been observed in patients with sarcoma, breast and ovarian carcinoma. Therefore this new drug is currently under intense investigation in several phase II and phase III clinical trials in cancer patients with a variety of neoplastic diseases.

Further detail on the use of ET-743, alone or in combination with another drug, for the treatment of the human body for cancer is given in WO 00 69441, incorporated herein by reference in its entirety.

Combination therapy using drugs with different mechanisms of action is an accepted method of treatment which helps prevent development of resistance by the treated tumor. In vitro activity of ET-743 in combination with other anticancer agents has been studied, see for example WO 02 36135, which is incorporated herein by reference in its entirety.

Docetaxel is an antineoplastic agent belonging to the taxoid family. It may be prepared by semisynthesis beginning with a precursor extracted from the renewable needle biomass of yew plants. The chemical name for docetaxel is (2R,3S)-N-carboxy-3-phenylisoserine, N-tert-butyl ester, 13-ester with 5β-20-epoxy-1,2α,4,7β,10β, 13α-hexahydroxytax-11-en-9-one 4-acetate 2-benzoate, trihydrate. Docetaxel has the following structural formula:

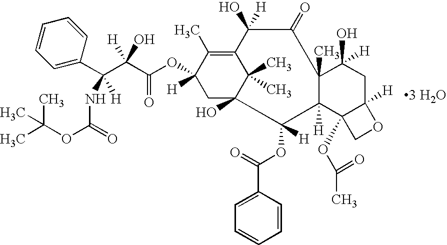

This antineoplastic agent acts by disrupting the microtubular network in cells that is essential for mitotic and interphase cellular functions. Docetaxel binds to free tubulin and promotes the assembly of tubulin into stable microtubules while simultaneously inhibiting their disassembly. This leads to the production of microtubule bundles without normal function and to the stabilization of microtubules, which results in the inhibition of mitosis in cells. It is indicated for the treatment of patients with locally advanced or metastatic breast cancer and non-small cell lung cancer after failure of prior chemotherapy.

Barrera H. et al. (see Proceedings of the American Association for Cancer Research, Volume 40, March 1999) reported a greater than additive interaction of ET743 and docetaxel against lung and breast tumor cell lines in in vitro assays.

It is an object of the invention to provide an efficacious treatment of cancer in humans based on the combination of ET-743 with docetaxel.

### BRIEF DESCRIPTIONS OF THE DRAWINGS

FIG. 1. Treatment duration at each dose level.

FIG. 2 and FIG. 3. Pharmacokinetic parameters of trabectedin and docetaxel following coadministration in patients with advanced malignancies.

### SUMMARY OF THE INVENTION

This invention relates to combination products, combination drug treatments and methods for treating patients afflicted with cancer.

In accordance with one aspect, the invention provides a combination therapy for the treatment of cancer in humans which comprises administering ET-743 and docetaxel, using a cyclical dosing protocol. Typical dosing protocols for the combination therapy are provided. The administration of ET-743 in combination with docetaxel in humans in accordance with the methods and compositions of this invention is tolerable and provides antitumor activity at the dosage and regimens given.

In addition, this inventions provides a method of treating cancer in humans, which method comprises administering ET-743 and docetaxel in a specific sequence and with a predetermined cycle.

Additionally, this invention further provides the use of ET-743 in the preparation of a medicament for carrying out the method of treatment. Also provided is the use of docetaxel in the preparation of a medicament for carrying out the method of treatment. The use of ET-743 and docetaxel in the preparation of a medicament for carrying out the method of treatment is also provided by this invention.

### DETAILED DESCRIPTION OF THE INVENTION

ET-743 is a natural compound with the following structure:

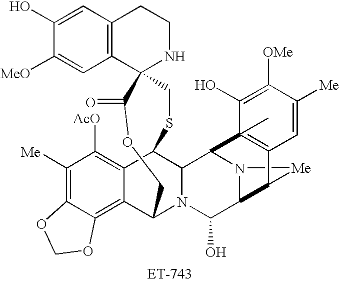

The term “ET-743” is intended here to cover any pharmaceutically acceptable salt, ester, solvate, hydrate or any other compound which, upon administration to the patient is capable of providing (directly or indirectly) the compound as described herein. However, it will be appreciated that non-pharmaceutically acceptable salts also fall within the scope of the invention since these may be useful in the preparation of pharmaceutically acceptable salts. The preparation of salts and prodrugs and derivatives can be carried out by methods known in the art. ET-743 for use in accordance of this invention may be obtained as a natural product by isolation and purification from Ecteinascidia turbinata as described in available reference material. Alternatively, ET743 may be prepared by a hemisynthetic or synthetic process, see for example WO 00 69862 and WO 01 87895, which are both incorporated herein by reference.

ET-743 may be supplied and stored as a sterile lyophilized product, consisting of ET-743 and excipient in a formulation adequate for therapeutic use, in particular a formulation containing sucrose and a phosphate salt buffered to an adequate pH. In other formulations, ET-743 in the form of a sterile lyophilized product is provided with mannitol and a phosphate salt buffered to an adequate pH. Further guidance on ET-743 formulations is given in WO 2006 046079 which is incorporated herein by reference in its entirety.

It has surprisingly been found that the combination of ET743 and docetaxel using a cyclical dosing protocol can lead to an increased anti-tumour efficacy in humans. The increased anti-tumour efficacy is in comparison to treatments using ET743 and docetaxel alone. In addition, it has been found that the combination of docetaxel and ET-743 is well tolerated to an extent in which both drugs may be administrated at full, or near full, therapeutic doses for prolonged periods of time. Further information regarding the dosage, schedules and administration of ET-743 alone or in combination is given in WO 00 69441, WO 02 36135, WO 03 039571, WO 2004 105761, WO 2005 039584, WO 2005 049031, WO 2005 049030, WO 2005 049029 and PCT/GB2005/050189, which are incorporated by reference herein in their entirety.

In one aspect, the invention is directed to the use of ET-743 in the preparation of a medicament for an effective treatment of the human body for cancer by combination therapy employing ET-743 with docetaxel.

In another aspect, the invention is directed to the use of docetaxel in the preparation of a medicament for an effective treatment of the human body for cancer by combination therapy employing docetaxel with ET-743.

The term “combination” as used throughout the specification, is meant to encompass the administration of the therapeutic agents in the same or separate pharmaceutical formulations, and at the same time or at different times.

In a further aspect, the present invention is directed to a method of treating the human body for cancer comprising administering an effective therapeutic amount of ET-743 in combination with an effective therapeutic amount of docetaxel.

The invention also provides a method of treating the human body for cancer comprising administering an effective therapeutic amount of docetaxel in combination with an effective therapeutic amount of ET-743.

In another aspect, the present invention is directed to a medical kit for administering ET-743 in combination with docetaxel, comprising a supply of ET-743 in dosage units for at least one cycle, wherein the dosage unit contains the appropriate amount of ET-743 for the treatments defined and a pharmaceutically acceptable carrier, and printed instructions for administering ET-743 according to a dosing schedule.

In another aspect, the present invention is directed to a medical kit for administering docetaxel in combination with ET-743, comprising a supply of docetaxel in dosage units for at least one cycle, wherein the dosage unit contains the appropriate amount of docetaxel for the treatments defined and a pharmaceutically acceptable carrier, and printed instructions for administering docetaxel according to a dosing schedule.

In another aspect, the present invention is directed to a medical kit for administering ET-743 in combination with docetaxel, comprising a supply of ET-743 in dosage units for at least one cycle, wherein the dosage unit contains the appropriate amount of ET-743 for the treatments defined and a pharmaceutically acceptable carrier, and printed instructions for administering ET-743 according to a dosing schedule. The kit also comprises a supply of docetaxel in dosage units for at least one cycle, wherein the dosage unit contains the appropriate amount of docetaxel for the treatments defined and a pharmaceutically acceptable carrier, and printed instructions for administering docetaxel according to a dosing schedule.

Administration of the compositions of this method is preferably by intravenous injection. Administration can be carried out continuously or periodically within the maximum tolerated dose (MTD). Throughout the specification, MTD is intended to relate to the highest dose at which less than one third of the subjects in a dose-level cohort experience dose limiting toxicity (DLT).

ET-743 and docetaxel may be provided as separate medicaments for administration at the same time or at different times. Preferably, ET-743 and docetaxel are provided as separate medicaments for administration at different times. When administered separately and at different times, either ET-743 or docetaxel may be administered first; however, it is preferable to administer docetaxel followed by ET743. Separating the administration of ET-743 and docetaxel by one hour is preferred.

Typical infusion times are up to 72 hours, more preferably 1-24 hours, with 1-6 hours most preferred. When docetaxel and ET-743 are provided as separate medicaments for administration at different times, the infusion times for each may differ.

Infusion times for docetaxel are generally up to 6 hours, more preferably 1-3 hours, with about 1 hour most preferred. Infusion times for ET-743 are generally up to 24 hours, more preferably about 1, about 3 or about 24 hours. Short infusion times which allow treatment to be carried out without an overnight stay in hospital are especially desirable.

It will be appreciated that the correct dosage of the compositions of this aspect of the invention will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. All dosages are expressed in milligrams (mg) per square metre (m²) of body surface area. Since in this method of the invention docetaxel and ET-743 are used in combination, the dosage of each is adjusted to provide the optimum clinical response.

In the present method of the invention, dosages of docetaxel of up to 120 mg/m² are used, more preferably 50-100 mg/m², with 60-75 mg/m² most preferred. Doses of about 60 mg/m², about 65 mg/m², about 70 mg/m² or about 75 mg/m² are particularly preferred, with about 60 mg/m² or about 75 mg/m² most preferred.

Dosages of ET-743 of up to 1.5 mg/m² are used, more preferably 0.4-1.3 mg/m², even more preferably 1.1-1.3 mg/m². Doses of about 0.4 mg/m², about 0.5 mg/m², about 0.6 mg/m², about 0.7 mg/m², about 0.8 mg/m², about 0.9 mg/m², about 1.0 mg/m², about 1.1 mg/m², about 1.2 mg/m², or about 1.3 mg/m² are particularly preferred, with about 1.1 mg/m² or about 1.3 mg/m² most preferred.

Illustrative embodiments of this invention are provided with dosages of docetaxel and ET-743 that are within the ranges given herein, each selected independently from the other within the respective dosage ranges. Illustrative embodiments of this invention are provided with dosages of about 60 mg/m² of docetaxel and about 1.1 mg/m² of ET-743, and about 60 mg/m² of docetaxel and about 1.3 mg/m² of ET-743. Additional illustrative embodiments of this invention are provided with dosages of about 75 mg/m² of docetaxel and about 1.3 mg/m² of ET-743.

According to a preferred embodiment of this aspect of the invention, 60-75 mg/m² of docetaxel are administered intravenously followed by up to 1.3 mg/m² ET-743, also administered intravenously. The docetaxel is preferably administered over an infusion time of up to 6 hours, more preferably 1-2 hours, most preferably about 1 hour. The ET-743 is preferably administered over an infusion time of about 1, about 2, about 3 or about 24 hours, most preferably the ET-743 is administered over about 3 hour.

According to a further preferred embodiment of this aspect of the invention, 60-75 mg/m² of docetaxel are administered intravenously followed by 0.4-1.3 mg/m² ET-743, also administered intravenously. The docetaxel is preferably administered over an infusion time of up to 6 hours, more preferably 1-2 hours, most preferably about 1 hour. The ET-743 is preferably administered over an infusion time of about 1, about 2, about 3 or about 24 hours, most preferably the ET-743 is administered over about 3 hour.

According to a further preferred embodiment of this aspect of the invention, 60-75 mg/m² of docetaxel are administered intravenously followed by 1.1-1.3 mg/m² ET-743, also administered intravenously. The docetaxel is preferably administered over an infusion time of up to 6 hours, more preferably 1-2 hours, most preferably about 1 hour. The ET-743 is preferably administered over an infusion time of about 1, about 2, about 3 or about 24 hours, most preferably the ET-743 is administered over about 3 hour.

According to a further preferred embodiment of this aspect of the invention, about 60 mg/m² of docetaxel are administered intravenously followed by about 1.1 mg/m² ET-743, also administered intravenously. The docetaxel is preferably administered over an infusion time of up to 6 hours, more preferably 1-2 hours, most preferably about 1 hour. The ET-743 is preferably administered over an infusion time of about 1, about 2, about 3 or about 24 hours, most preferably the ET-743 is administered over about 3 hour.

According to a further preferred embodiment of this aspect of the invention, about 60 mg/m² of docetaxel are administered intravenously followed by about 1.3 mg/m² ET-743, also administered intravenously. The docetaxel is preferably administered over an infusion time of up to 6 hours, more preferably 1-2 hours, most preferably about 1 hour. The ET-743 is preferably administered over an infusion time of about 1, about 2, about 3 or about 24 hours, most preferably the ET-743 is administered over about 3 hour.

According to a further preferred embodiment of this aspect of the invention, about 75 mg/m² of docetaxel are administered intravenously followed by about 1.1 mg/m² ET-743, also administered intravenously. The docetaxel is preferably administered over an infusion time of up to 6 hours, more preferably 1-2 hours, most preferably about 1 hour. The ET-743 is preferably administered over an infusion time of about 1, about 2, about 3 or about 24 hours, most preferably the ET-743 is administered over about 3 hour.

According to a further preferred embodiment of this aspect of the invention, about 75 mg/m² of docetaxel are administered intravenously followed by about 1.3 mg/m² ET-743, also administered intravenously. The docetaxel is preferably administered over an infusion time of up to 6 hours, more preferably 1-2 hours, most preferably about 1 hour. The ET-743 is preferably administered over an infusion time of about 1, about 2, about 3 or about 24 hours, most preferably the ET-743 is administered over about 3 hour.

The administration of the combination is performed in cycles in accordance with the method of the invention. Intravenous infusions of docetaxel and ET-743 are given to the patients typically every 3 weeks, allowing for a resting phase in each cycle in which the patients recover. The preferred duration of each cycle is typically of 3 to 4 weeks; multiple cycles can be given as needed. Dose delays and/or dose reductions and schedule adjustments are performed as needed depending on individual patient tolerance to treatments.

According to a particularly preferred embodiment, every 3 weeks, up to 120 mg/m² of docetaxel are administered to a patient over an infusion time of about 1 hour followed, after about 1 hour of rest, by administration of up to 1.5 mg/m² of ET-743 over an infusion time of about 3 hours.

According to a further particularly preferred embodiment, every 3 weeks, 50-100 of docetaxel are administered to a patient over an infusion time of about 1 hour followed, after about 1 hour of rest, by administration of about 0.4-1.3 mg/m² of ET-743 over an infusion time of about 3 hours.

According to a further particularly preferred embodiment, every 3 weeks, 50-100 of docetaxel are administered to a patient over an infusion time of about 1 hour followed, after about 1 hour of rest, by administration of about 1.1-1.3 mg/m² of ET-743 over an infusion time of about 3 hours.

According to a further particularly preferred embodiment, every 3 weeks, 60-75 mg/m² of docetaxel are administered to a patient over an infusion time of about 1 hour followed, after about 1 hour of rest, by administration of about 0.4-1.3 mg/m² of ET-743 over an infusion time of about 3 hours.

According to a further particularly preferred embodiment, every 3 weeks, 60-75 of docetaxel are administered to a patient over an infusion time of about 1 hour followed, after about 1 hour of rest, by administration of about 1.1-1.3 mg/m² of ET-743 over an infusion time of about 3 hours.

According to a further particularly preferred embodiment, every 3 weeks, about 60 mg/m² or about 75 mg/m² of docetaxel are administered to a patient over an infusion time of about 1 hour followed, after about 1 hour of rest, by administration of about 1.1 mg/m² or about 1.3 mg/m² of ET-743 over an infusion time of about 3 hours.

According to a further particularly preferred embodiment, every 3 weeks, about 60 mg/m² or about 75 mg/m² of docetaxel are administered to a patient over an infusion time of about 1 hour followed, after about 1 hour of rest, by administration of about 1.3 mg/m² of ET-743 over an infusion time of about 3 hours.

According to a further particularly preferred embodiment, every 3 weeks, about 60 mg/m² or about 75 mg/m² of docetaxel are administered to a patient over an infusion time of about 1 hour followed, after about 1 hour of rest, by administration of about 1.1 mg/m² of ET-743 over an infusion time of about 3 hours.

According to a further particularly preferred embodiment, every 3 weeks, about 60 mg/m² of docetaxel are administered to a patient over an infusion time of about 1 hour followed, after about 1 hour of rest, by administration of about 1.1 mg/m² of ET-743 over an infusion time of about 3 hours.

According to a further particularly preferred embodiment, every 3 weeks, about 75 mg/m² of docetaxel are administered to a patient over an infusion time of about 1 hour followed, after about 1 hour of rest, by administration of about 1.1 mg/m² of ET-743 over an infusion time of about 3 hours.

According to a further particularly preferred embodiment, every 3 weeks, about 60 mg/m² of docetaxel are administered to a patient over an infusion time of about 1 hour followed, after about 1 hour of rest, by administration of about 1.3 mg/m² of ET-743 over an infusion time of about 3 hours.

According to a further particularly preferred embodiment, every 3 weeks, about 75 mg/m² of docetaxel are administered to a patient over an infusion time of about 1 hour followed, after about 1 hour of rest, by administration of about 1.3 mg/m² of ET-743 over an infusion time of about 3 hours.

The above dosage embodiments are also applicable to a 4 week infusion schedule.

By using a dosing regimen in accordance with that used in this preferred embodiment, it has been found that the combination is well tolerated when both drugs are administrated at full, or near full, therapeutic doses for prolonged periods of time.

Premedication and supportive medication can be given. As noted in the incorporated article by Kesteren, the combination of ET-743 with dexamethasone gives unexpected advantages, since it has a role in prophylaxis of hepatic toxicity. Therefore, it is preferred to administer dexamethasone to the patient, typically at around the time of infusing ET-743. For example, it is preferred to begin administration of dexamethasone one day prior to the start of ET-743 administration, and to continue administering dexamethasone throughout the period of ET-743 administration ending at least 1 day after ET-743 administration is completed. The administration of dexamethasone can be extended, for example to one or more days preceding or following the administration of ET-743. Likewise, it is also possible to administer dexamethasone as premedication for docetaxel. Further options include administration of filgrastim (G-CSF) as supportive medication for docetaxel and/or ET-743 and 5-HT₃ antagonist as premedication or supportive medication for ET-743. Preadministration of both dexamethasone and G-CSF as supportive medication are particularly preferred in the method of the invention.

Depending on the type of tumor and the development stage of the disease, the treatments of the invention are useful in promoting tumor regression, in stopping tumor growth and/or in preventing metastasis. In particular, the method of the invention is suited for human patients, especially those who are relapsing or refractory to previous chemotherapy. First line therapy is also envisaged.

Preferably, the combination therapy is used according to the above schedules and dosages for the treatment of sarcoma, osteosarcoma, ovarian cancer, breast cancer, melanoma, pancreatic cancer, gastric adenocarcinoma, colorectal cancer, mesothelioma, renal cancer, endometrial cancer, head and neck carcinoma, prostate cancer and lung cancer (NSCLC and SCLC). Most preferably the patients are sarcoma patients, especially those with a soft tissue sarcoma. Ovarian cancer and breast cancer are also preferably suited for the combination therapy.

It has been surprisingly determined that if ET-743 and docetaxel are administered in combination in accordance with the method of this invention, both chemotherapeutic agents may be administered at the same full doses, or near full, as if they were administered as single agents. Surprisingly, the toxicity of these two agents when combined does not prevent the use of full therapeutic doses of each agent. Accordingly, the same full dose of ET-743, i.e., 1.3 mg/m² over 3 hours every 21 days, that would be administered if ET-743 was administered as a single agent may be administered with the full dose of docetaxel as it would be used in clinical practice. i.e., 60-100 mg/m² over 1 hour every 21 days, if administered as a single agent.

Accordingly, in another aspect, the present invention is directed to a method for maximising the tolerated dose of ET-743 in a treatment of the human body for cancer comprising administering an effective therapeutic amount of ET-743 in combination with docetaxel.

Example 1 shows the results of a study to evaluate the MTD of ET-743 in combination with 75 mg/m² or 60 mg/m² of docetaxel, together with results of phase I trials.

This invention therefore provides methods of treatment, the use of the compounds in the preparation of a composition for treatment of cancer and related embodiments. The present invention also extends to the compositions of the invention for use in a method of treatment.

The present invention also relates to pharmaceutical preparations including a pharmaceutically acceptable carrier, which contain as active ingredient a compound or compounds of the invention, as well as the processes for their preparation.

In another aspect, the present invention also provides a pharmaceutical composition comprising an effective therapeutic amount of ET-743, or a pharmaceutically acceptable salt thereof, and a pharmaceutically acceptable carrier, for use in the procedures and methods as defined herein.

In a further aspect, the present invention also provides a pharmaceutical composition comprising an effective therapeutic amount of docetaxel, or a pharmaceutically acceptable salt thereof, and a pharmaceutically acceptable carrier, for use in the procedures and methods as defined herein.

In addition, the present invention also provides a pharmaceutical composition comprising an effective therapeutic amount of ET-743, or a pharmaceutically acceptable salt thereof, an effective therapeutic amount of docetaxel, or a pharmaceutically acceptable salt thereof, and a pharmaceutically acceptable carrier, for use in the procedures and methods as defined herein.

In another aspect, the invention further provides for the use of ET-743, or a pharmaceutically acceptable salt thereof, in the preparation of a composition for use in the procedures and methods as defined herein.

In a related aspect, the invention further provides for the use of docetaxel, or a pharmaceutically acceptable salt thereof, in the preparation of a composition for use in the procedures and methods as defined herein.

In a further aspect, the invention also provides for the use of ET-743, or a pharmaceutically acceptable salt thereof, and docetaxel, or a pharmaceutically acceptable salt thereof, in the preparation of a composition for use in the procedures and methods as defined herein.

In another aspect, the invention further provides for the use of ET-743, or a pharmaceutically acceptable salt thereof, for the manufacture of a medicament for the treatment of cancer, in combination therapy with docetaxel.

In a related aspect, the invention further provides for the use of docetaxel, or a pharmaceutically acceptable salt thereof, for the manufacture of a medicament for the treatment of cancer, in combination therapy with ET-743.

In a related aspect, the invention further provides for the use of ET-743, or a pharmaceutically acceptable salt thereof, in combination with docetaxel, or a pharmaceutically acceptable salt thereof, for the manufacture of a medicament for the treatment of cancer.

In another aspect, the invention further provides for the use of ET-743, or a pharmaceutically acceptable salt thereof, for the treatment of cancer, in combination therapy with docetaxel.

In a related aspect, the invention further provides for the use of docetaxel, or a pharmaceutically acceptable salt thereof, for the treatment of cancer, in combination therapy with ET-743.

In another aspect, the invention further provides for the use of ET-743, or a pharmaceutically acceptable salt thereof, in combination with docetaxel, or a pharmaceutically acceptable salt thereof, for the treatment of cancer.

In another aspect, the invention further provides for the use of ET-743, or a pharmaceutically acceptable salt thereof, as a medicament, in combination therapy with docetaxel.

In a related aspect, the invention further provides for the use of docetaxel, or a pharmaceutically acceptable salt thereof, as a medicament, in combination therapy with ET-743.

In another aspect, the invention further provides for the use of ET-743, or a pharmaceutically acceptable salt thereof, in combination with docetaxel, or a pharmaceutically acceptable salt thereof, as a medicament.

In another aspect, the invention further provides for the use of ET-743, or a pharmaceutically acceptable salt thereof, as a medicament for the treatment of cancer, in combination therapy with docetaxel.

In a related aspect, the invention further provides for the use of docetaxel, or a pharmaceutically acceptable salt thereof, as a medicament for the treatment of cancer, in combination therapy with ET-743.

In another aspect, the invention further provides for the use of ET-743, or a pharmaceutically acceptable salt thereof, in combination with docetaxel, or a pharmaceutically acceptable salt thereof, as a medicament for the treatment of cancer.

In another aspect, the invention further provides for the use of ET-743, or a pharmaceutically acceptable salt thereof, as a medicament formulated to be provided in a dosage and/or schedule as defined herein for the treatment of cancer, in combination therapy with docetaxel.

In a related aspect, the invention further provides for the use of docetaxel, or a pharmaceutically acceptable salt thereof, as a medicament formulated to be provided in a dosage and/or schedule as defined herein for the treatment of cancer, in combination therapy with ET-743.

In another aspect, the invention further provides for the use of ET-743, or a pharmaceutically acceptable salt thereof, as a medicament formulated to be provided in a dosage and/or schedule as defined herein, in combination with docetaxel, or a pharmaceutically acceptable salt thereof, as a medicament formulated to be provided in a dosage and/or schedule as defined herein for the treatment of cancer.

In another aspect, the invention further provides for the use of ET-743, or a pharmaceutically acceptable salt thereof, for the manufacture of a medicament formulated to be provided in a dosage and/or schedule as defined herein for the treatment of cancer, in combination therapy with docetaxel.

In a related aspect, the invention further provides for the use of docetaxel, or a pharmaceutically acceptable salt thereof, for the manufacture of a medicament formulated to be provided in a dosage and/or schedule as defined herein for the treatment of cancer, in combination therapy with ET-743.

In another aspect, the invention further provides for the use of ET-743, or a pharmaceutically acceptable salt thereof, and docetaxel, or a pharmaceutically acceptable salt thereof, for the manufacture of a medicament formulated to be provided in a dosage and/or schedule as defined herein for the treatment of cancer.

In another aspect, the invention further provides for the formulation of one or more dosage units of ET-743, a pharmaceutically acceptable salt thereof, or a pharmaceutical composition thereof, said dosage units formulated to be provided in a dosage and/or schedule as defined herein for the treatment of cancer, in combination therapy with docetaxel.

In a related aspect, the invention further provides for the formulation of one or more dosage units of docetaxel, a pharmaceutically acceptable salt thereof, or a pharmaceutical composition thereof, said dosage units formulated to be provided in a dosage and/or schedule as defined herein for the treatment of cancer, in combination therapy with ET-743.

In another aspect, the invention further provides for the formulation of one or more dosage units of ET-743, a pharmaceutically acceptable salt thereof, or a pharmaceutical composition thereof, and one or more dosage units of docetaxel, a pharmaceutically acceptable salt thereof, or a pharmaceutical composition thereof, said dosage units formulated to be provided in a dosage and/or schedule as defined herein for the treatment of cancer.

In another aspect, the invention further provides for the use of ET-743, or a pharmaceutically acceptable salt thereof, in the preparation of a composition, formulated to be provided in a dosage and/or schedule as defined herein for use in the procedures and methods as defined herein, in combination therapy with docetaxel.

In a related aspect, the invention further provides for the use of docetaxel, or a pharmaceutically acceptable salt thereof, in the preparation of a composition, formulated to be provided in a dosage and/or schedule as defined herein for use in the procedures and methods as defined herein, in combination therapy with ET-743.

In another aspect, the invention further provides for the use of ET-743, or a pharmaceutically acceptable salt thereof, in the preparation of a composition, and docetaxel, or a pharmaceutically acceptable salt thereof, in the preparation of a composition, both formulated to be provided in a dosage and/or schedule as defined herein for use in the procedures and methods as defined herein.

In another aspect, the invention further provides a medicament, dosage unit(s), formulation or composition of ET-743, or a pharmaceutically acceptable salt thereof, specifically configured to the dosages and/or schedules given herein, the ET-743 being given in combination therapy with docetaxel. This specific configuration is carried out during the preparation process of the final medicament, and is not part of the actions carried out by the doctor when treating the patient.

In a related aspect, the invention further provides a medicament, dosage unit(s), formulation or composition of docetaxel, or a pharmaceutically acceptable salt thereof, specifically configured to the dosages and/or schedules given herein, the docetaxel being given in combination therapy with ET-743. This specific configuration is carried out during the preparation process of the final medicament, and is not part of the actions carried out by the doctor when treating the patient.

In another aspect, the invention further provides a medicament, dosage unit(s), formulation or composition of ET-743, or a pharmaceutically acceptable salt thereof, specifically configured to the dosages and/or schedules given herein, together with a medicament, dosage unit(s), formulation or composition of docetaxel, or a pharmaceutically acceptable salt thereof, specifically configured to the dosages and/or schedules given herein. This specific configuration is carried out during the preparation process of the final medicament, and is not part of the actions carried out by the doctor when treating the patient.

In a further aspect, the invention provides a dosage unit(s), medicament, formulation or composition comprising ET-743, or a pharmaceutically acceptable salt thereof, specifically adapted to be administered in the dosages and/or schedules given herein, the ET-743 being given in combination therapy with docetaxel.

In a related aspect, the invention provides a dosage unit(s), medicament, formulation or composition comprising docetaxel, or a pharmaceutically acceptable salt thereof, specifically adapted to be administered in the dosages and/or schedules given herein, the docetaxel being given in combination therapy with ET-743.

In a further aspect, the invention provides a dosage unit(s), medicament, formulation or composition comprising ET-743, or a pharmaceutically acceptable salt thereof, and a dosage unit(s), medicament, formulation or composition comprising docetaxel, or a pharmaceutically acceptable salt thereof, specifically adapted to be administered in the dosages and/or schedules given herein.

In a further aspect, the invention provides for the use of ET-743, or a pharmaceutically acceptable salt thereof, in a medicament for the treatment of cancer wherein the medicament is configured for administration at the dosages and/or schedules given herein, the ET-743 being given in combination therapy with docetaxel.

In a related aspect, the invention provides for the use of docetaxel, or a pharmaceutically acceptable salt thereof, in a medicament for the treatment of cancer wherein the medicament is configured for administration at the dosages and/or schedules given herein, the docetaxel being given in combination therapy with ET-743.

In a further aspect, the invention provides for the use of ET-743, or a pharmaceutically acceptable salt thereof, and docetaxel, or a pharmaceutically acceptable salt thereof, in a medicament for the treatment of cancer wherein the medicament is configured for administration at the dosages and/or schedules given herein.

In a further aspect, the invention provides for the use of ET-743, or a pharmaceutically acceptable salt thereof, for the manufacture of a medicament for the treatment of cancer wherein the medicament is configured for administration at the dosages and/or schedules given herein, the ET-743 being given in combination therapy with docetaxel.

In a related aspect, the invention provides for the use of docetaxel, or a pharmaceutically acceptable salt thereof, for the manufacture of a medicament for the treatment of cancer wherein the medicament is configured for administration at the dosages and/or schedules given herein, the docetaxel being given in combination therapy with ET-743.

In a further aspect, the invention provides for the use of ET-743, or a pharmaceutically acceptable salt thereof, and docetaxel, or a pharmaceutically acceptable salt thereof, for the manufacture of a medicament for the treatment of cancer wherein the medicament is configured for administration at the dosages and/or schedules given herein.

The following example further illustrates the invention. It should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### Examples of the Invention

Throughout the Example Ecteinascidin 743 (ET-743) is also referred to as Trabectedin.

### Example 1

A phase I trial combining docetaxel and trabectedin was performed. The objective of this study was to determine the maximum tolerated dose (MTD) of trabectedin in combination with docetaxel (60 mg/m² or 75 mg/m²) administered every 21 days. Additional objectives were to evaluate the safety profile of this combination of drugs and to evaluate the pharmacokinetics of trabectedin and docetaxel when given in combination. The maximum tolerated dose (MTD) relates to the highest dose at which less than one third of the subjects in a dose-level cohort experienced dose-limiting toxicity (DLT).

Subjects enrolled in this clinical trial were adult (age≧18 years) patients with a metastatic or unresectable malignancy for which standard curative or palliative measures do not exist or are no longer effective. Entry criteria included Eastern Cooperative Oncology Group (ECOG) Performance Status (PS) score of 0 or 1, clinically adequate recovery from acute toxicity of prior therapies, adequate hepatic and renal function and no prior exposure to trabectedin.

We designed a dose finding trial with a fixed docetaxel dose of 60 mg/m² or 75 mg/m² administered intravenously over one hour, followed by one of six trabectedin doses (0.4, 0.6, 0.75, 0.9, 1.1, and 1.3 mg/m²) administered intravenously over 3 hours, with one hour of rest between the administration of docetaxel and ET-743. This treatment was repeated every 21 days. Table 1 shows the dose escalation applied in this trial, using the standard “3+3” dose escalation guidelines. Restricted patients received ≦1 prior chemotherapy regimen whereas unrestricted patients had no limit. In addition, protocol amendment after dose level 2a allowed administration of primary prophylactic G-CSF.

TABLE 1
Dose Escalation Schedule

| | | | Trabectedin |
|---|---|---|---|
| Dose Level | # of Patients | Docetaxel (mg/m²)^{‡} | (mg/m²)^{‡} |
| 1 (unrestricted) | 6 | 60 | 0.4 |
| 2 (unrestricted) | 4 | 60 | 0.6 |
| 2a (restricted) | 5 | 60 | 0.6 |
| 2b (restricted, G-CSF) | 3 | 60 | 0.6 |
| 3 (restricted, G-CSF) | 3 | 60 | 0.75 |
| 4 (restricted, G-CSF) | 3 | 60 | 0.9 |
| 5 (restricted, G-CSF) | 3 | 60 | 1.1 |
| 6 (restricted, G-CSF) | 7 | 60 | 1.3 |

^{‡}If toxicity in Cycle 2 or beyond, docetaxel dose reduction to 50 or 60 mg/m² (from 60 or 75 mg/m², respectively) and then trabectedin reduction if second occurrence. Third occurrence or DLT in Cycle 1 required discontinuation.

Two additional levels, level 6a and level 7, with a dosage of 60 mg/m² of docetaxel and 1.3 mg/m² of trabectedin (level 6a), and 75 mg/m² of docetaxel and 1.3 mg/m² of trabectedin (level 7) were also evaluated. Four unrestricted patients were enrolled for dose level 6a and 3 restricted patients were enrolled for dose level 7. In both levels, patients also received G-CSF as primary prophylactic.

As supportive measures, dexamethasone was administered as a 3-day course premedication, staffing the day prior to each cycle (total of 5 oral doses plus a single intravenous dose prior to docetaxel infusion on Day 1). In addition, patients received filgrastim (G-CSF) as supportive medication: originally the administration of G-CSF was only permitted after Cycle 1, however protocol was amended allowing administration of primary prophylactic G-CSF in cohorts 2b and higher. Finally, other treatments such as transfusions, antibiotics, and supplemental antiemetics were also administered as necessary.

Dose-limiting toxicities (DLTs) were defined as follows:
Absolute neutrophil count (ANC)<500 μL for >5 days or in conjunction with fever or sepsis
Platelet count<25,000/μL
Grade 3/4 nonhematologic toxicity (except nausea/vomiting, unless occurring despite appropriate antiemetic prophylaxis) or grade 3 transaminitis lasting >1 week
Delay of initiation of a subsequent course of therapy by >3 weeks

Thirty four patients, 10 with sarcoma, 4 with prostate cancer, 3 with head and neck carcinoma, 2 with NSCLC, 2 with SCLC, 1 with colorectal cancer, 1 with gastric cancer, 1 with ovarian cancer, 1 with pancreatic cancer, 1 with renal cancer, and 8 more with other types of cancers have been treated. Eleven patients of 34 had a Performance Status (PS) of 0 (fully active, able to carry on all pre-disease performance without restriction) (Table 2).

TABLE 2
Demographic and clinical characteristics

| | Total |
|---|---|
| | N = 34 |
| Sex | |
| Female | 14 |
| Male | 20 |
| Age | |
| 18-65 | 22 |
| >65 | 12 |
| Median | 56.0 |
| Range | 31-72 |
| ECOG score | |
| 0 | 11 |
| 1 | 23 |
| Histology | |
| Colorectal | 1 |
| Gastric | 1 |
| Head & neck | 3 |
| NSCLC | 2 |
| Ovary | 1 |
| Pancreatic | 1 |
| Prostate | 4 |
| Renal | 1 |
| Sarcoma | 10 |
| SCLC | 2 |
| Other | 8 |
| Previous therapy | |
| Systemic | 24 |
| Surgery | 28 |
| Radiotherapy | 17 |

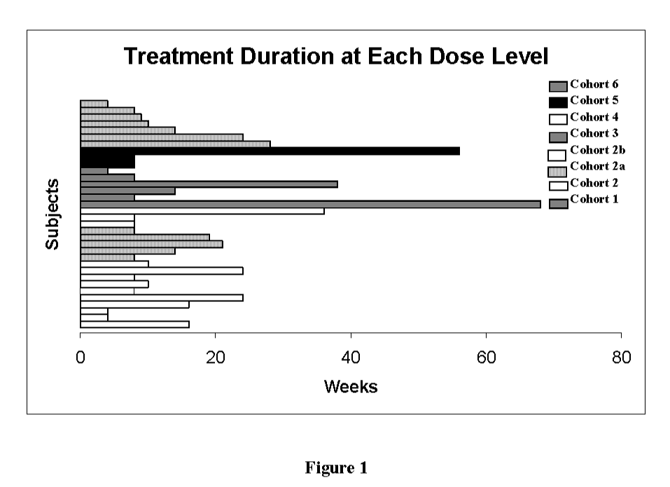

Table 3 and FIG. 1 show the total treatment duration in each dose level.

TABLE 3
Exposure to Treatment: Total Treatment Duration

| | | | | |
|---|---|---|---|---|
| Level 1 | Level 2 | Level 2a | Level 2b | Level 3 |

Treatment duration (weeks)

| | | | | | |
|---|---|---|---|---|---|
| Median | 9.00 | 9.07 | 12.14 | 6.14 | 12.14 |
| Range | 3.00-24.00 | 6.00-25.00 | 6.00-20.14 | 6.00-33.00 | 6.29-67.14 |

Number of cycles

| | | | | | |
|---|---|---|---|---|---|
| Median | 3 | 2.5 | 4 | 2 | 4 |
| Range | 1-8 | 2-8 | 2-6 | 2-10 | 2-22 |

| | | | |
|---|---|---|---|
| Level 4 | Level 5 | Level 6 | Total |

Treatment duration (weeks)

| | | | | |
|---|---|---|---|---|
| Median | 6.00 | 6.00 | 9.43 | 9.07 |
| Range | 3.00-37.14 | 6.00-55.00 | 0-28.00 | 3.00-97.00 |

Number of cycles

| | | | | |
|---|---|---|---|---|
| Median | 2 | 2 | 3 | 3 |
| Range | 1-11 | 2-17 | 1-9 | 1-22 |

Table 4 shows the relative dose intensity in each dose level.

TABLE 4
Exposure to Treatment: Overall Relative Dose Intensity

| | | | | |
|---|---|---|---|---|
| Level 1 | Level 2 | Level 2a | Level 2b | Level 3 |

Trabectedin

| | | | | | |
|---|---|---|---|---|---|
| Median | 1.00 | 0.87 | 0.83 | 0.98 | 0.96 |
| Range | 0.93-1.03 | 0.55-1.00 | 0.62-1.00 | 0.93-1.02 | 0.95-1.14 |

Docetaxel

| | | | | | |
|---|---|---|---|---|---|
| Median | 1.00 | 0.93 | 0.91 | 0.98 | 0.95 |
| Range | 0.93-1.03 | 0.61-1.00 | 0.75-1.00 | 0.93-1.01 | 0.67-0.96 |

| | | | |
|---|---|---|---|
| Level 4 | Level 5 | Level 6 | Total |

Trabectedin

| | | | | |
|---|---|---|---|---|
| Median | 1.00 | 0.93 | 0.96 | 0.97 |
| Range | 0.68-1.00 | 0.85-1.00 | 0.92-1.02 | 0.55-1.14 |

Docetaxel

| | | | | |
|---|---|---|---|---|
| Median | 1.00 | 0.92 | 0.96 | 0.96 |
| Range | 0.77-1.00 | 0.85-1.00 | 0.92-1.00 | 0.61-1.03 |

A total of 6 DLTs were reported, which are shown in table 5.

TABLE 5
Dose-Limiting Toxicity

| | Level 1 | Level 2 | Level 2a | Level 6 | Total |
|---|---|---|---|---|---|
| Total no. with DLT | 1 | 2 | 2 | 1 | 6 |
| ANC < 500/μL > 5 d | 0 | 2 | 1 | 0 | 3 |
| Febrile neutropenia | 1 | 1 | 1 | 0 | 3 |
| Neutropenia | 0 | 1 | 1 | 0 | 2 |
| Fatigue | 0 | 0 | 0 | 1 | 1 |

In addition to the above mentioned DLTs, 1 out of 3 patients treated at level 7 developed a DLT, which was related to grade 3 fatigue.

Worst on-treatment drug related grade 3 and 4 toxicities were related to neutropenia, leukopenia, transaminases elevation and fatigue. Table 6 shows the frequently reported drug-related Grade 3/4 adverse events in at least 2 patients (≧5% of subjects). The adverse events reported at any time from first treatment dose up to 30 days after the last treatment dose are included. In order to define the toxicity grade, NCI Common Toxicity Criteria, version 2.0, was used.

TABLE 6
Grade 3/4 Drug-Related Adverse Events in at Least 2 Patients (≧5%)

| | Level 1 | Level 2 | Level 2a | Level 2b | Level 3 |
|---|---|---|---|---|---|
| | n = 6 | n = 4 | n = 5 | n = 3 | n = 3 |
| Total with grade | 5 | 4 | 5 | 1 | 1 |
| 3/4 | | | | | |
| Neutropenia | 5 | 4 | 5 | 1 | 1 |
| Leukopenia | 0 | 0 | 4 | 1 | 0 |
| Elevated ALT | 0 | 0 | 0 | 0 | 1 |
| Fatigue | 0 | 0 | 0 | 0 | 0 |

| | Level 4 | Level 5 | Level 6 | Total |
|---|---|---|---|---|
| | n = 6 | n = 4 | n = 5 | N = 34 |
| Total with grade 3/4 | 1 | 0 | 3 | 20 (59) |
| Neutropenia | 0 | 0 | 2 | 18 (53) |
| Leukopenia | 0 | 0 | 0 | 5 (15) |
| Elevated ALT | 1 | 0 | 1 | 3 (9) |
| Fatigue | 0 | 0 | 2 | 2 (6) |

In addition, Table 7 shows the most common adverse events reported. Adverse events reported at any time from first treatment dose to within 30 days of last treatment dose are included. Incidence is based on the number of patients.

TABLE 7
Most Common Adverse Events (≧30%)

| | Level 1 | Level 2 | Level 2a | Level 2b | Level 3 |
|---|---|---|---|---|---|
| | n = 6 | n = 4 | n = 5 | n = 3 | n = 3 |
| Total with AE | 6 | 4 | 5 | 3 | 3 |
| Fatigue | 3 | 4 | 3 | 1 | 3 |
| Nausea | 2 | 2 | 5 | 1 | 2 |
| Neutropenia | 5 | 4 | 5 | 1 | 1 |
| Anemia | 2 | 1 | 3 | 2 | 0 |
| Cough | 3 | 1 | 1 | 1 | 1 |
| Diarrhea | 2 | 2 | 1 | 0 | 1 |
| Vomiting | 0 | 1 | 3 | 2 | 1 |
| Constipation | 2 | 2 | 2 | 0 | 1 |
| Alopecia | 2 | 2 | 2 | 1 | 1 |

| | Level 4 | Level 5 | Level 6 | Total |
|---|---|---|---|---|
| | n = 3 | n = 3 | n = 7 | N = 34 |
| Total with AE | 3 | 3 | 6 | 33 (97) |
| Fatigue | 3 | 1 | 5 | 23 (68) |
| Nausea | 2 | 3 | 3 | 20 (59) |
| Neutropenia | 0 | 0 | 2 | 18 (53) |
| Anemia | 1 | 1 | 3 | 13 (38) |
| Cough | 1 | 2 | 3 | 13 (38) |
| Diarrhea | 1 | 3 | 2 | 12 (35) |
| Vomiting | 0 | 3 | 2 | 12 (35) |
| Constipation | 0 | 2 | 2 | 11 (32) |
| Alopecia | 1 | 0 | 2 | 11 (32) |

There were no deaths within 30 days of last dose due to disease progression.

One patient, with peritoneal cancer, had a complete response. Seventeen patients (4 with prostate cancer, 4 with sarcoma, 2 with SCLC and one each liposarcoma, malignant mesothelioma pleura, stromatosis, gallbladder, gastric and ovarian cancer, and one unknown) had stable disease (Table 8).

TABLE 8
Best Overall Response

| | Level 1 | Level 2 | Level 2a | Level 2b | Level 3 |
|---|---|---|---|---|---|
| Complete response | 0 | 0 | 1 | 0 | 0 |
| Peritoneal | 0 | 0 | 1 | 0 | 0 |
| Stable disease | 3 | 2 | 2 | 1 | 2 |
| Gallbladder | 0 | 0 | 1 | 0 | 0 |
| Gastric | 0 | 0 | 0 | 1 | 0 |
| Liposarcoma | 0 | 1 | 0 | 0 | 0 |
| Malignant | 0 | 0 | 0 | 0 | 0 |
| mesothelioma (L) | | | | | |
| pleura | | | | | |
| Ovary | 1 | 0 | 0 | 0 | 0 |
| Prostate | 0 | 0 | 1 | 0 | 0 |
| Sarcoma | 1 | 0 | 0 | 0 | 1 |
| SCLC | 1 | 0 | 0 | 0 | 0 |
| Stromatosis | 0 | 1 | 0 | 0 | 0 |
| Unknown | 0 | 0 | 0 | 0 | 1 |
| Progressive disease | 1 | 2 | 2 | 2 | 1 |
| Head & neck | 0 | 0 | 1 | 1 | 1 |
| Non small cell | 0 | 0 | 0 | 0 | 0 |
| carcinoma | | | | | |
| (unknown primary) | | | | | |
| NSCLC | 0 | 0 | 0 | 0 | 0 |
| Pancreatic | 0 | 1 | 0 | 0 | 0 |
| Renal | 0 | 0 | 0 | 0 | 0 |
| Sarcoma | 1 | 1 | 1 | 1 | 0 |
| Not reported | 2 | 0 | 0 | 0 | 0 |
| Colorectal | 1 | 0 | 0 | 0 | 0 |
| Sarcoma | 0 | 0 | 0 | 0 | 0 |
| Submandibular | 1 | 0 | 0 | 0 | 0 |
| gland | | | | | |

| | Level 4 | Level 5 | Level 6 | Total |
|---|---|---|---|---|
| Complete response | 0 | 0 | 0 | 1 |
| Peritoneal | 0 | 0 | 0 | 1 |
| Stable disease | 1 | 1 | 5 | 17 |
| Gallbladder | 0 | 0 | 0 | 1 |
| Gastric | 0 | 0 | 0 | 1 |
| Liposarcoma | 0 | 0 | 0 | 1 |
| Malignant mesothelioma (L) pleura | 0 | 0 | 1 | 1 |
| Ovary | 0 | 0 | 0 | 1 |
| Prostate | 1 | 0 | 2 | 4 |
| Sarcoma | 0 | 1 | 1 | 4 |
| SCLC | 0 | 0 | 1 | 2 |
| Stromatosis | 0 | 0 | 0 | 1 |
| Unknown | 0 | 0 | 0 | 1 |
| Progressive disease | 1 | 2 | 1 | 12 |
| Head & neck | 0 | 0 | 1 | 3 |
| Non small cell carcinoma | 0 | 0 | 1 | 1 |
| (unknown primary) | | | | |
| NSCLC | 1 | 1 | 0 | 2 |
| Pancreatic | 0 | 0 | 0 | 1 |
| Renal | 0 | 1 | 0 | 1 |
| Sarcoma | 0 | 0 | 0 | 4 |
| Not reported | 1 | 0 | 1 | 4 |
| Colorectal | 0 | 0 | 0 | 1 |
| Sarcoma | 1 | 0 | 1 | 2 |
| Submandibular gland | 0 | 0 | 0 | 1 |

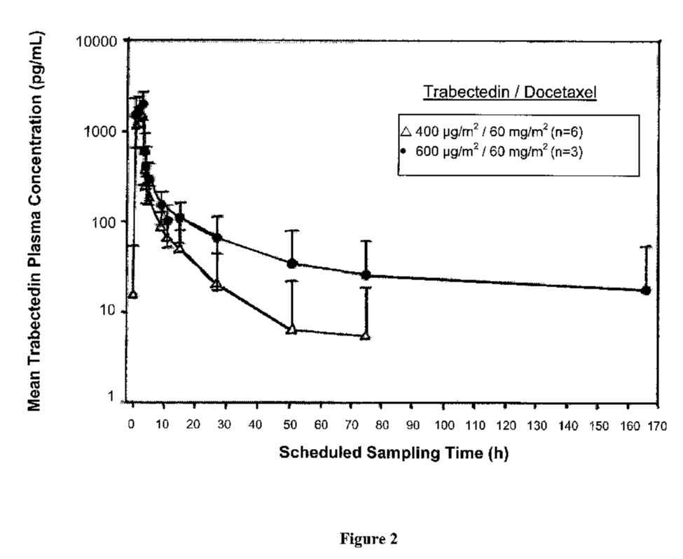

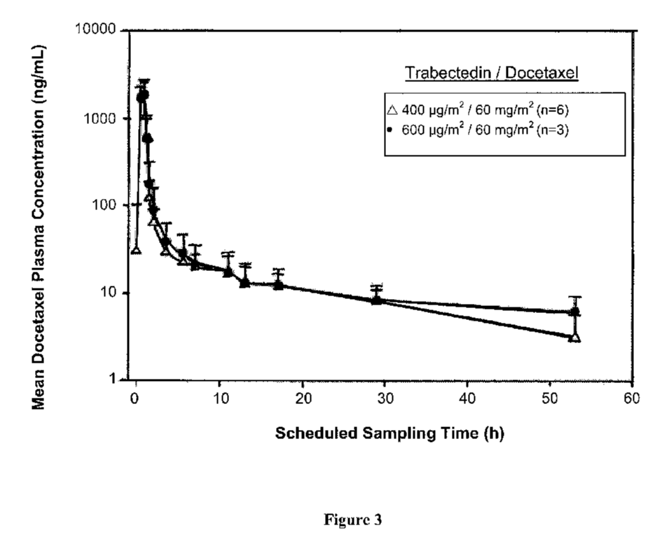

No pharmacokinetic interaction was observed following administration of trabectedin and docetaxel (Table 9a, FIG. 2 and Table 9b, FIG. 3).

TABLE 9a
Mean (SD) Pharmacokinetic Parameters of Trabectedin Following
Coadministration with 60 mg/m²of Docetaxel

Trabectedin Dose

| | 400 μg/m² (n = 6) | 600 μg/m² (n = 3) |
|---|---|---|
| CL (L/h) | 127 (61) | 101 (46) |
| Vₛₛ (L) | 1179 (1402) | 2754 (1946) |
| Terminal t_{1/2} (h)^{a} | 11.4 (3.3-100) | 37.5 (11.7-98.8) |

^{a}median (range)

TABLE 9b
Mean (SD) Pharmacokinetic Parameters of Docetaxel (60 mg/m²)
Following Coadministration with Trabectedin

Trabectedin Dose

| | 400 μg/m² (n = 6) | 600 μg/m² (n = 4) |
|---|---|---|
| CL (L/h) | 48.9 (15.3) | 47.8 (15) |
| Vₛₛ (L) | 405 (179) | 900 (339) |
| Terminal t_{1/2} (h)^{a} | 19.9 (6.7-23) | 38.2 (33.8-42.8) |

^{a}median (range)

From this study we conclude that administration of trabectedin and docetaxel with G-CSF support is safe and well tolerated. In addition, it has been demonstrated that this combination is well tolerated when both drugs are administered at full (or near full) therapeutic doses for prolonged periods of time.

Preliminary data suggest activity for the trabectedin docetaxel combination in patients with advanced cancers, with 1 patient achieving a complete response and 17 maintaining stable disease.

In addition, it has been shown that the pharmacokinetics of docetaxel was overtly not adversely impacted by concomitant administration of trabectedin.

1. A method of treating the human body for cancer comprising administering an effective therapeutic amount of ET-743 in combination with an effective therapeutic amount of docetaxel.

2. The method according to claim 1, wherein ET-743 and docetaxel are administered at full or near full therapeutic doses as if they were administered as single agents.

3. The method according to claim 1 or 2, wherein ET-743 and docetaxel are administered sequentially.

4. The method according to claim 3, wherein docetaxel is first administered followed by ET-743.

5. The method according to claim 3 or 4, wherein ET-743 and docetaxel are administered by intravenous infusion, and wherein the infusion time for ET-743 is up to 24 hours and the infusion time of docetaxel is up to 6 hours.

6. The method according to claim 5, wherein the infusion time for ET-743 is about 3 hours.

7. The method according to claim 5 or 6, wherein the infusion time for docetaxel is about 1 hour.

8. The method according to any preceding claim, wherein ET-743 and docetaxel are administered once every 3 or 4 weeks.

9. The method according to claim 8, wherein ET-743 and docetaxel are administered once every 3 weeks.

10. The method according to claim 9, wherein ET-743 and docetaxel are administered in the same day, and wherein docetaxel is first administered followed, after about 1 hour of rest, by the administration of ET-743.

11. The method according to any preceding claim, wherein ET-743 is administered at a dose between about 0.4 and about 1.3 mg/m².

12. The method according to claim 11, wherein ET-743 is administered at a dose of about 1.1 mg/m² or about 1.3 mg/m².

13. The method according to any preceding claim, wherein docetaxel is administered at a dose between about 50 and about 100 mg/m².

14. The method according to claim 13, wherein docetaxel is administered at a dose of about 60 mg/m² or about 75 mg/m².

15. The method according to claim 14, wherein docetaxel is administered at a dose of about 60 mg/m² over an infusion time of about 1 hour followed by the administration of ET-743 at a dose of about 1.3 mg/m² over an infusion time of about 3 hours.

16. The method according to claim 14, wherein docetaxel is administered at a dose of about 60 mg/m² over an infusion time of about 1 hour followed by the administration of ET-743 at a dose of about 1.1 mg/m² over an infusion time of about 3 hours.

17. The method according to claim 14, wherein docetaxel is administered at a dose of about 75 mg/M2 over an infusion time of about 1 hour followed by the administration of ET-743 at a dose of about 1.1 mg/m² over an infusion time of about 3 hours.

18. The method according to claim 14, wherein docetaxel is administered at a dose of about 75 mg/m² over an infusion time of about 1 hour followed by the administration of ET-743 at a dose of about 1.3 mg/m² over an infusion time of about 3 hours.

19. The method according to any preceding claim, wherein the method further comprises administering filgrastim.

20. The method according to any preceding claim, in which the patient is relapsing or refractory to previous chemotherapy.

21. The method according to any preceding claim, in which the patient has a cancer selected from sarcoma, osteosarcoma, ovarian cancer, breast cancer, melanoma, pancreatic cancer, gastric adenocarcinoma, colorectal cancer, mesothelioma, renal cancer, endometrial cancer, head and neck carcinoma, prostate cancer and lung cancer.

22. The use of ET-743 in the preparation of a medicament for a method according to any of the preceding claims.

23. The use of docetaxel in the preparation of a medicament for a method according to any of claims 1 to 21.

24. A medical kit for administering ET-743 in combination with docetaxel, comprising a supply of ET-743 in dosage units for at least one cycle, wherein the dosage unit contains the appropriate amount of ET-743 for a method according to any of claims 1 to 21 and a pharmaceutically acceptable carrier, and printed instructions for administering ET-743 in accordance with said method.

25. A medical kit for administering docetaxel in combination with ET-743, comprising a supply of docetaxel in dosage units for at least one cycle, wherein the dosage unit contains the appropriate amount of docetaxel for a method according to any of claims 1 to 21 and a pharmaceutically acceptable carrier, and printed instructions for administering docetaxel in accordance with said method.
